(19) United States
(12) Patent Application Publication
(10) Pub. No.: US 2012/0308553 A1
(43) Pub. Date: Dec. 6, 2012

(54) ORAL MEDICATION FOR THE TREATMENT OF HEMORRHOIDS AND METHOD OF USE

(76) Inventor: Joseph P. Thornton, Las Vegas, NV (US)

(21) Appl. No.: 13/588,910

(22) Filed: Aug. 17, 2012

Related U.S. Application Data
(62) Division of application No. 12/547,294, filed on Aug. 25, 2009, now abandoned.

(51) Int. Cl. A61K 38/48; A61P 1/00; A61P 29/00

(52) U.S. Cl. 424/94.65

(57) ABSTRACT

A natural supplement in the form of an orally ingested tablet for treatment of hemorrhoids. Bromelain and Psyllium (e.g., Psyllium husk powder) are the primary active ingredients in the natural supplement tablet. Optionally, Witch Hazel, in the form of Witch Hazel leaf, for example, may be added to the composition. Secondary ingredients include Dicalcium Phosphate, Cellulose, Croscarmellose Sodium, Stearic Acid and Silicon Dioxide. Based on case studies the natural supplement outperforms over-the-counter and prescribed hemorrhoid medications.

### RELATED APPLICATIONS

This application is a divisional of U.S. patent application Ser. No. 12/547,294, filed Aug. 29, 20009 which is related to co-pending U.S. patent application Ser. No. 12/547,193, filed Aug. 25, 2009 and U.S. patent application Ser. No. 12/547,241, filed Aug. 25, 2009, all of which are incorporated herein in their entirety.

### FIELD OF THE INVENTION

The embodiments of the present invention relate to an oral composition for the treatment of hemorrhoids.

### BACKGROUND

Hemorrhoids are swollen veins in the anal canal. Millions of people suffer from hemorrhoids and a majority of adults will suffer from hemorrhoids at one point in their life. Treatments for hemorrhoids range from surgery to topical ointments. Surgery includes rubber band ligation, cryosurgery and surgical hemorrhoidectomy. Non-surgical procedures include the application of heat, use of lasers or electric current to create scar tissue, or sclerotheraphy. Medicinal treatments include ointments and creams such as Preparation H® which may be applied directly or via medicated wipes and the like. Despite the various treatments, hemorrhoids continue to flourish.

Thus, there exists a need for natural treatment to more reliably and efficiently treating hemorrhoids.

### SUMMARY

Accordingly, one embodiment of the present invention is a supplement tablet or pill including at least one or more anti-inflammatory agents and a laxative agent. In one embodiment, the supplement tablet includes one or both of Bromelain and Hamamelis Virginiana (commonly known as Witch Hazel) as the anti-inflammatory agent and Psylluim as a laxative. Other ingredients are compounded with the Bromelain and/or Witch Hazel and Psylluim to form a tablet or pill as the delivery vehicle.

In one embodiment, for active symptoms, the supplement tablet is taken three times per day for 10 days. For lesser symptoms, the natural supplement tablet may be taken once per day. As evidenced by the case studies described below, the use of the natural supplement tablet reduces dramatically the swelling, pain, bleeding and other symptoms associated with hemorrhoids.

Other variations, embodiments and features of the present invention will become evident from the following detailed description, drawings and claims.

### BRIEF DESCRIPTION OF THE DRAWINGS

FIG. 1 illustrates an ingredient list for a natural supplement tablet according to one embodiment of the present invention;

FIG. 2 illustrates a flow chart detailing one treatment protocol utilizing the natural supplement tablet according to the embodiments of the present invention; and

FIG. 3 illustrates a kit container designed to hold a container of the soaking agent along with the tablets and gel/ointment as described in the referenced co-pending applications.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

A natural treatment for hemorrhoids has benefits for both the individual with the ailment and society as a whole. The benefit to society is a decreased need for surgery, prescription drugs and other unnecessary unnatural treatments. The individual suffering from hemorrhoids benefits by being able to take a single natural tablet one to three times per day. Over-the-counter topical Preparation H® utilizes mineral oil (protectant), petrolatum (protectant), Phenylephrine (vasoconstrictor) and shark liver oil (protectant). Over-the-counter topical Tucks® utilizes a combination of mineral oil (protectant), Pramoxine HCL (pain relief) and Zinc Oxide (protectant). Over-the-counter topical Balneol is essentially a moisturizer. Therefore, the active ingredients in most over-the-counter topical applications are utilized primarily to protect and treat pain in the area affected by hemorrhoids but do not effectively treat or prevent the hemorrhoids. Prescription drugs, like topical Analpram, utilize Pramoxine HCL (pain relief) and Hydrocortisone (steroidal anti-inflammatory), but still fail to treat hemorrhoids as well as the embodiments of the present invention. Another benefit to the over-the-counter natural tablet is that is can be used in conjunction with the gel/ointment described in co-pending patent application Ser. No. 12/547,241 and/or the soaking agent described in co-pending patent application Ser. No. 12/547,193. Any of the treatments alone or in combination facilitate the avoidance of expensive prescription drugs. In many instances today, patients are not able to afford prescription drugs. This is true for uninsured and insured patients given large deductibles and countless uncovered prescriptions.

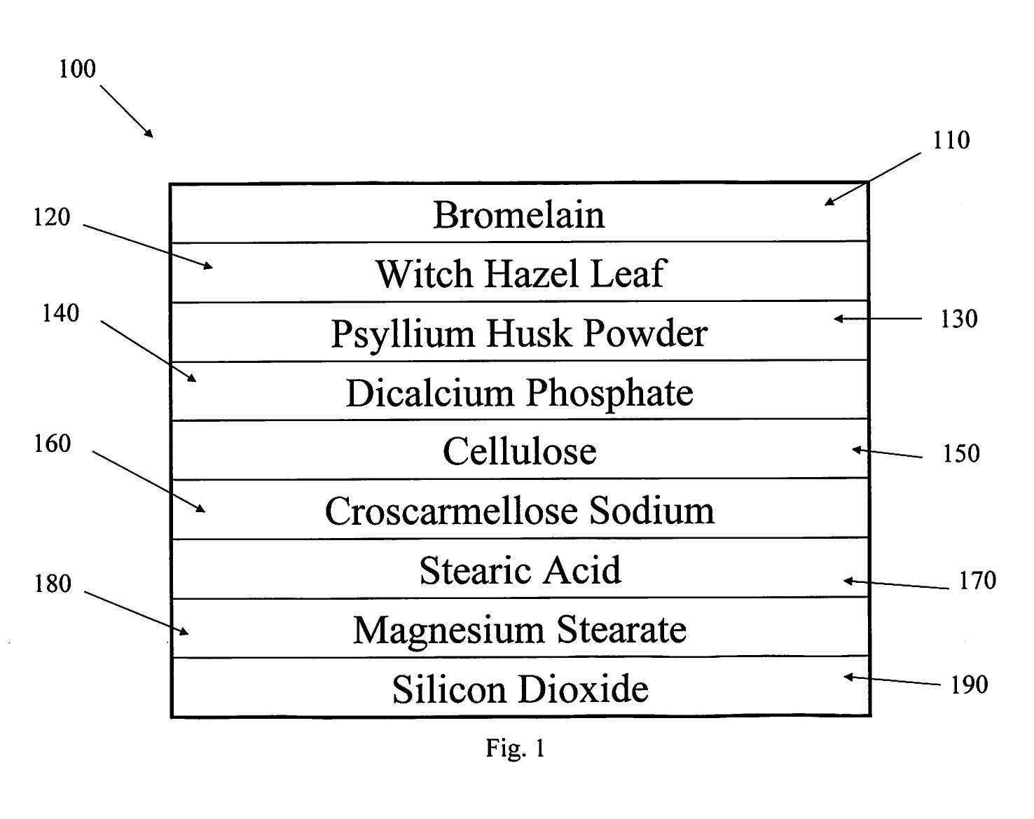

FIG. 1 depicts an ingredient list 100 for a first embodiment of the present invention. The three possible primary active ingredients comprise Bromelain 110, Witch Hazel leaf 120 and Psyllium husk powder 130. The Bromelain 110 and Witch Hazel leaf 120 have an anti-inflammatory effect while the Psyllium husk powder acts as a bulk laxative. When combined, the Bromelain 110, Witch Hazel leaf 120 and Psyllium husk powder 130 have a synergistic effect as evidenced by the case studies detailed below. Alternatively, Bromelain alone can be used as the anti-inflammatory agent.

Bromelain is a mixture of protein-digesting (proteolytic) enzymes found in pineapples. Bromelain, which is derived from the stem and juice of the pineapple, was first isolated from the pineapple plant in the late 1800s. Witch Hazel is a low growing shrub native to North America. Witch Hazel has many uses including as an anti-inflammatory, astringent, toner and numbing agent. Psyllium husk is the covering of seeds grown on the plant, Plantago Psyllium, which flourishes in the Middle East. Psyllium husk has been known to provide dietary fiber thus acting as a controlled bulk forming laxative.

In a first embodiment, as depicted in the ingredient list 100 a tablet or tablet according to the embodiments of the present invention comprises 500 mg of Bromelain 1200 GDU 110, 100 mg of Witch Hazel leaf 120 and 100 mg of Psyllium husk powder 130. Additional, secondary ingredients in the tablet include Dicalcium Phosphate 140, Cellulose 150, Croscarmellose Sodium 160, Stearic Acid 170, Magnesium Stearate 180 and Silicon Dioxide 190. Dicalcium Phosphate 140 is also known as Calcium Monohydrogen Phosphate and is a dibasic Calcium Phosphate. Dicalcium Phosphate 140 is a known tablet agent. Cellulose 150 (e.g., Hydroxyethyl Cellulose) is a nonionic polymer used as a thickening agent. Croscarmellose Sodium 160 is an excipient that acts as an inactive substance used as a carrier for the other active ingredients in the tablet. Stearic Acid 170 and Magnesium Stearate 180 (aka Octadecanoic Acid) are saturated fatty acids, and Silicon Dioxide 190 is another excipient. Those skilled in the art will recognize that other ingredients having the same properties may be used in place of, or in addition to, the aforementioned secondary ingredients. Also, certain of the listed secondary ingredients may be removed while maintaining the effectiveness of the tablet. The ingredients are combined into a pill or tablet form using conventional manufacturing techniques.

In one embodiment, for active swelling, the tablets in accordance with the specifications listed above, are to be taken 3 times daily for 10 days. To handle flare ups, 1 tablet may be taken daily. Other treatment protocols may be possible with larger/smaller tablets.

Table 1 details exemplary case studies. Advantageously, the results of utilizing the tablets in accordance with the embodiments of the present invention have dramatic synergistic effects over usage of the active ingredients individually. In addition, a high majority of the patients expressed that the tablets outperformed other over-the-counter medications such as common ointments.

TABLE 1

| | | | | | | Tablet More | Tablet |
|---|---|---|---|---|---|---|---|
| | | | | Improvement | | Effective Than | Effectiveness |
| Age | Gender | Diagnosis | Symptoms | Time | OTC Products | OTC Meds? | 0-5 |
| 81 | Female | Hemorrhoids | Swelling, | 2 days | Tucks | Yes | 5 |
| | | | Bleeding & Pain | | | | |
| 60 | Female | Hemorrhoids | Swelling | 1 week | Prep H | Yes | 5 |
| 62 | Female | Hemorrhoids | Swelling | 2 weeks | Analpram | Yes | 4 |
| 57 | Male | Hemorrhoids | Itching | N/A | N/A | No | 1 |
| N/A | Male | Hemorrhoids | Swelling | days | Prep H | Yes | 5 |
| 78 | Male | Hemorrhoids | Bleeding & Pain | 2 weeks | Analpram | Yes | 3 |
| 80 | Male | Hemorrhoids | Swelling & Pain | 2 or more | Prep H | Yes | 5 |
| | | | | days | | | |
| 39 | Female | Hemorrhoids | Bleeding & | 10 days | Prep H | Yes | 5 |
| | | | Itching | | | | |
| 59 | Female | Hemorrhoids | Swelling | 2 days | Prep H | Yes | |
| N/A | N/A | Hemorrhoids | Swelling | 7 days | Prep H | Yes | 5 |
| N/A | N/A | Hemorrhoids | Swelling, Bleeding | 2 weeks | Prep H | Yes | 4 |
| | | | & Pain | | | | |
| N/A | N/A | Hemorrhoids | Swelling | 7 days | Prep H | Yes | 5 |
| 47 | Male | Hemorrhoids | Swelling, Bleeding, | 2 days | Tucks, Prep H | Yes | 5 |
| | | | Pain & Itching | | | | |
| 63 | Female | Hemorrhoids | Swelling, Bleeding, | 2 days | Tucks, Prep H | Yes | 5 |
| | | | Pain & Itching | | | | |
| 45 | Female | Hemorrhoids | Bleeding & Pain | 3 days | Tucks, Prep H | Yes | 5 |
| N/A | N/A | Hemorrhoids | Swelling & Bleeding | 2 weeks | N/A | Yes | 5 |
| N/A | Male | Hemorrhoids | Bleeding | 2 or more | Prep H | Yes | 5 |
| | | | | days | | | |
| 61 | N/A | Hemorrhoids | Swelling | 1 week | N/A | Yes | 4 |
| 80 | Female | Hemorrhoids | Swelling | 3 weeks | N/A | Yes | 3 |
| 80 | Male | Hemorrhoids | Swelling & Pain | 10 days | Prep H | Yes | 5 |
| 66 | Male | Hemorrhoids | Swelling | 7 days | Prep H | Yes | 5 |
| 66 | N/A | Hemorrhoids | Swelling | 7 days | Prep H | Yes | 4 |

Table 1 lists the age and gender of a majority of patients. Also shown in Table 1 are the diagnosis, symptoms, time for results, over-the-counter medications used by the patients, each patient's opinion of whether the tablets have better results than the over-the-counter medications and a rating score of effectiveness associated with the tablets. Table 1 evidences that the tablets treat the typical symptoms namely swelling, bleeding, pain and itching associated with hemorrhoids. Positive results from the tablets were felt in a range of 2 days to 3 weeks. And, except for 1 patient, each patient opined that the tablets treated hemorrhoids better than over-the-counter medications (e.g., Preparation H® (Prep H), Tucks®, Analpram and Balneol®). In addition, the patients rated the treatment as an average of 4.45 out of 5 on the ratings scale. Such feedback, including the average rating, evidences a dramatic effect associated with the ingredients in the tablet form.

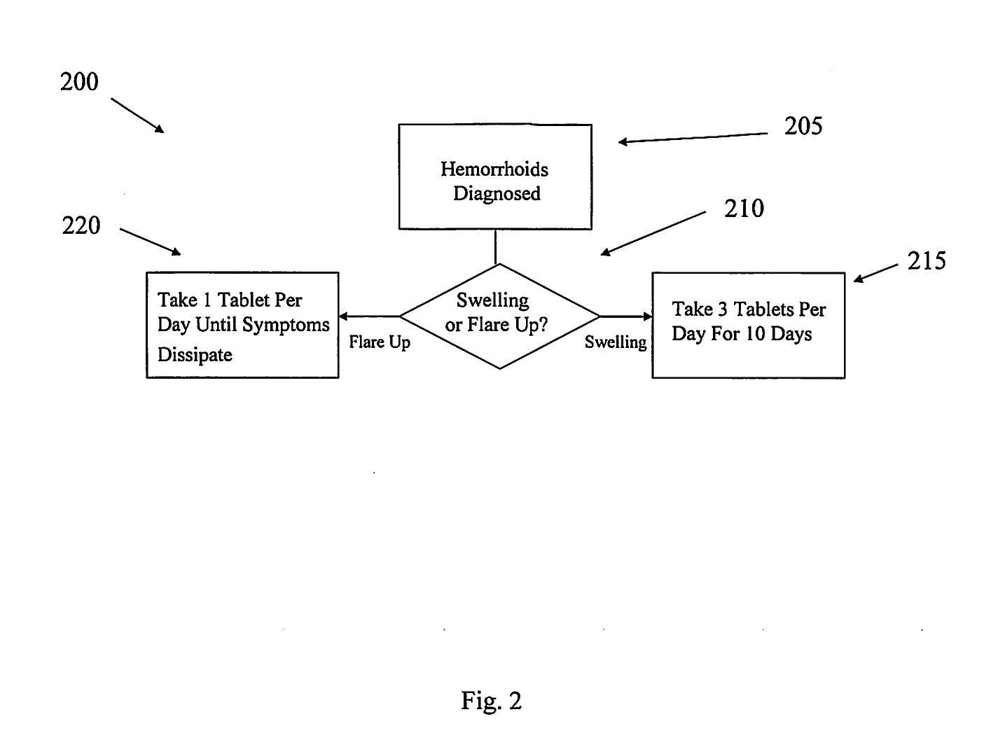

FIG. 2 shows a flow chart 200 detailing a method of treating hemorrhoids using the tablets. At 205, hemorrhoids is diagnosed. At 210, it is determined whether the patient has active swelling or a flare up. If the patient has active swelling, at 215, the patient is instructed to take 3 tablets per day for 10 days. If the patient has a flare up, at 220, the patient is instructed to take a tablet daily until the hemorrhoid symptoms dissipate.

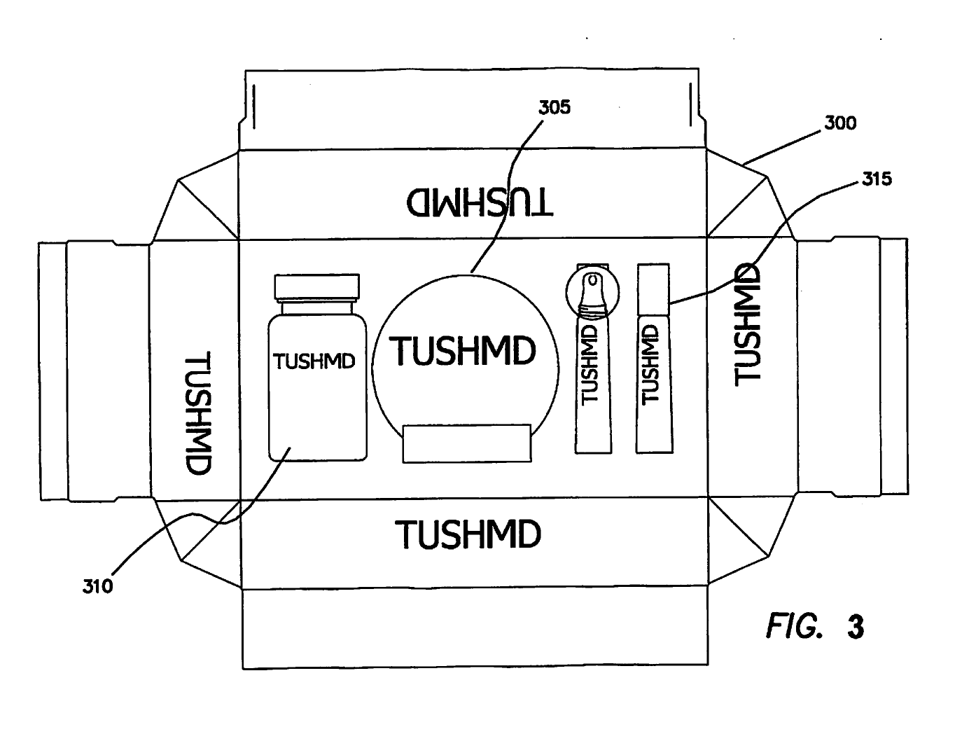

FIG. 3 shows a form 300 for constructing a box to hold a kit comprising individual containers for each of the soaking agent 305 (described in co-pending patent application Ser. No. 12/547,193), tablets 310 and gel/ointment 315 (described in co-pending patent application Ser. No. 12/547,241). Separately or alone, the soaking agent, tablets and gel/ointment effectively treat hemorrhoids. The treatment protocols for each delivery vehicle remain the same when each is used in combination as when used individually. In other words, all three treatment protocols can be used simultaneously without any negative side effects.

Although the invention has been described in detail with reference to several embodiments, additional variations and modifications exist within the scope and spirit of the invention as described and defined in the following claims.

I claim:

1. An orally ingested tablet comprising:
Bromelain; and
an anti-inflammatory agent.

2. The orally ingested tablet of claim 1 wherein said anti-inflammatory agent is Witch hazel leaf.

3. The orally ingested tablet of claim 1 further comprising Psyllium husk powder.

4. The orally ingested tablet of claim 1 further comprising one or more of the following ingredients: Dicalcium Phosphate, Cellulose, Croscarmellose Sodium, Stearic Acid and Silicon Dioxide.
